(19) United States
(12) Patent Application Publication
(10) Pub. No.: US 2024/0217910 A1
(43) Pub. Date: Jul. 4, 2024

(54) PROCESSES FOR PRODUCING BIOMONOMERS AND PRECURSORS FOR SAME

(71) Applicant: UOP LLC, Des Plaines, IL (US)

(72) Inventors: Yili Shi, Buffalo Grove, IL (US); Veronica G. Deak, Chicago, IL (US); Kristen E. Allaire, Chicago, IL (US); Joel T. Walenga, Lake Zurich, IL (US)

(21) Appl. No.: 18/506,340

(22) Filed: Nov. 10, 2023

Related U.S. Application Data
(60) Provisional application No. 63/477,860, filed on Dec. 30, 2022.

(51) Int. Cl. C07C 51/15; C07C 51/42

(52) U.S. Cl. CPC C07C 51/15; C07C 51/42

(57) ABSTRACT

Processes for producing biomonomers and precursors for producing same. A carboxylation reaction is conducted between an aromatic hydrocarbon, such as a furoate, and a dicarboxylated alkali salt. The reaction produces a carboxylated aromatic hydrocarbon and a decarboxylated alkali salt. The carboxylated aromatic hydrocarbon can be separated and used to produce biomonomers like furan dicarboxylate methyl ester and furan dicarboxylic acid. The decarboxylated alkali salt may be regenerated.

### RELATED APPLICATIONS

This application claims priority to U.S. Provisional Patent Application Ser. No. 63/477,860, filed Dec. 30, 2022, the entirety of which is incorporated herein by reference.

### FIELD OF THE INVENTION

This invention relates generally to process for the production of aromatic carboxylic acid compounds including furan dicarboxylic acid and furan dicarboxylate methyl ester from biomass.

### BACKGROUND OF THE INVENTION

Recently, processes have been developed for producing aromatic carboxylic acids and esters from sugars produced from biomass. These aromatic carboxylic acids and esters can be converted to dicarboxylates which can in turn be utilized to produce monomers like furan dicarboxylate methyl ester (FDME) and furan dicarboxylic acid (FDCA). As is known, these monomers are useful in making polymers and plastics and, since they are at least in part derived from biomass, may be referred to as biomonomers.

These processes are desirable because they provide for the production of the biomonomers as opposed to producing chemicals and monomers from fossil fuel sources. Additionally, the processes are desirable because they may consume carbon dioxide -which is considered a greenhouse gas.

These processes typically rely on a furoate carboxylation reaction with carbon dioxide and an alkali base. While generally effective for their intended purposes, such a reaction is difficult to optimize and thermal furoate decarboxylation to furan is a competing side reaction.

Thus, there is an ongoing desire and need to provide effective and efficient processes for producing biomonomers from biomass derived components and carbon dioxide.

### SUMMARY OF THE INVENTION

The present inventors have discovered an alternative furoate carboxylation reaction that occurs at lower temperatures to high conversion and produces less byproducts for downstream separation. Specifically, it was found that the reaction of a hydrocarbon having an aromatic ring with an alkali salt dicarboxylate produces a carboxylated aromatic compound and a decarboxylated alkali salt. Additionally, no extra alkali base reagent was needed in for the present carboxylate transfer reaction.

Therefore, the present invention may be characterized, in at least one aspect, as providing a process for conducting a carboxylate transfer reaction by: mixing an aromatic ring with a dicarboxylate alkali salt to form a mixture; and, heating the mixture in the presence of carbon dioxide to form a carboxylated aromatic compound and a decarboxylated alkali salt.

The aromatic ring may include a counter ion, and the alkali base, the counter ion, or both may be selected from a group consisting of: lithium, sodium, potassium, rubidium, cesium, and mixtures thereof.

The dicarboxylate alkali salt may be a 1, 3-dicarboxylate alkali salt.

The mixture may be heated to a temperature between 120° C. to 400° C. at a pressure up to 6,895 kPa (1,000 psi).

The mixture may be a slurry and the slurry may be formed in a hydrocarbon.

The carbon dioxide may be provided as bubbles which flow counter current.

The aromatic ring may be a furoate, and the dicarboxylate alkali salt may be malonate.

The process may also include regenerating the dicarboxylate alkali salt from the decarboxylated alkali salt.

The present invention may also be generally characterized as providing a process for producing a carboxylated aromatic compound by: passing an aromatic hydrocarbon and a dicarboxylate alkali salt to a vessel in a reaction zone to form a mixture; passing carbon dioxide into the vessel to contact the mixture; and, heating the mixture to form a carboxylated aromatic compound and a decarboxylated alkali salt.

The aromatic hydrocarbon may further include a counter ion, and the alkali base, the counter ion, or both may be selected from a group consisting of: lithium, sodium, potassium, rubidium, cesium, and mixtures thereof.

The dicarboxylate alkali salt may be a 1, 3-dicarboxylate alkali salt.

The aromatic hydrocarbon may be a furoate and the dicarboxylate alkali salt may be malonate.

The mixture may be heated to a temperature between 120° C. to 400° C. at a pressure up to 6,895 kPa (1,000 psi).

The mixture may be a slurry and the slurry may be formed in a hydrocarbon. The carbon dioxide may be provided as bubbles which flow counter current to the slurry.

The process may also include separating the carboxylated aromatic compound from the hydrocarbon forming the slurry. The process can also include recycling the hydrocarbon forming the slurry.

The process may include regenerating the dicarboxylate alkali salt from the decarboxylated alkali salt.

Additional aspects, embodiments, and details of the invention, all of which may be combinable in any manner, are set forth in the following detailed description of the invention.

### DETAILED DESCRIPTION OF THE INVENTION

As mentioned above, the present invention provides processes for conducting a carboxylate transfer reaction between a hydrocarbon with an aromatic ring and a dicarboxylate alkali salt. Preferably, the hydrocarbon with an aromatic ring is a furoate which is produced from a biomass. See, U.S. Pat. Nos. 7,572,925 and 8,772,515. As used herein “biomass” includes, but is not limited to, lignin, plant parts, fruits, vegetables, plant processing waste, wood chips, chaff, grain, grasses, corn, corn husks, weeds, aquatic plants, hay, paper, paper products, recycled paper and paper products, and any cellulose, lignin, or combinations thereof containing biological material or material of biological origin.

In general, the process mixes the aromatic hydrocarbon and the dicarboxylate alkali salt to form a mixture. This mixture is heated to reaction temperature in the presence of a carbon dioxide gas, potentially in a counter-current slurry-bubble column reactor. The hydrocarbon with an aromatic ring and the dicarboxylate alkali salt react and form a carboxylated aromatic compound and a decarboxylated alkali salt. The carboxylated aromatic compound can then be converted in subsequent chemicals steps into either biomonomers like FDCA or FDME. The decarboxylated alkali salt may be regenerated and recycled in the process.

With these general principles in mind, one or more embodiments of the present invention will be described with the understanding that the following description is not intended to be limiting.

Methods according to the present invention include forming a mixture between an aromatic hydrocarbon and a dicarboxylate alkali salt. The aromatic hydrocarbon is preferably a furoate. A furoate counter ion and the alkali of the dicarboxylate alkali salt may each, independently be lithium, sodium, potassium, rubidium, cesium, and mixtures thereof. The dicarboxylate may be a 1,3 di carboxylate, for example, dipotassium malonate.

The dicarboxylate alkali salt may be at a mole ratio of dicarboxylate alkali salt to aromatic hydrocarbon may be from about 1:1 to 2:1, 1:0.1 to 1:1, 1:0.1 to 1:0.5, or 0.1:1 to 1:1.

The mixture may be formed in a hydrocarbon oil such as a hydrocarbon material containing between 5 to 30 carbon atoms per molecule and having paraffinic and/or aromatic functional groups to form a slurry. In general, the hydrocarbon oil selected for the slurry will have negligible solubility to the aromatic hydrocarbon and the dicarboxylate alkali salt.

Additionally, carbon dioxide is provided to the mixture. For example, carbon dioxide can be provided as bubbles into the slurry. The bubbles may flow counter current to the flow of the slurry.

With the carbon dioxide, the mixture is heated to a temperature of between 120 to 400° C., 150 to 360° C., or between 270 to 330° C., at a pressure from about atmospheric up to 6,895 kPa (1,000 psi), or up to 4,826 kPa (700 psig), or up to 4,137 kPa (600 psig) and sufficient heat for a time sufficient to form a carboxylated aromatic compound and a decarboxylated alkali salt via a carboxylation reaction between the reagents. The reaction time is sufficient to produce the carboxylated aromatic compound is from about 1 second to 24 hours, 1 minute to 12 hours, 1 minute to 6 hours, or 1 minute to 1 hour. The process may be continuous, semi-batch or batch reaction process.

The carboxylated aromatic compounds that are produced can include terephthalic acid, naphthalic acid, thiophene dicarboxylic acid, pyridine dicarboxylic acid, carbazole dicarboxylic acid, and dibenzothiophene dicarboxylic acid. In particular, the carboxylated aromatic compound may be furan dicarboxylate, and specifically, furan-2,4-dicarboxylate and/or furan-2,5-dicarboxylate.

The carboxylated aromatic compound may be recovered by being separated from the slurry. The recovered carboxylated aromatic compound may be converted to FDME, FDCA, or both. In particular, the produced biomonomers may include one or more of furan-2,5-dicarboxylic acid, furan 2,4 dicarboxylic acid, dimethyl furan-2,5-dicarboxylate, dimethyl furan-2,4-dicarboxylate, and salts thereof. These biomonomers may converted in polymers as is known in the art.

After the carboxylated aromatic compound have been separated, the decarboxylated alkali salt may be regenerated and recycled.

Compared with existing reactions, the present reactions provide improved yield and do not require carboxylation reaction promoters.

### EXPERIMENTS

One (1) molar eq. K-furoate was mixture with 1.0 molar equivalent K2-malonate. The mixture was heated, in the presence of carbon dioxide, to 250° C. for 5 hours. A K-furoate conversion was found to be 55% (mol), and a K2-FDCA yield was found to be 33 wt %.

### SPECIFIC EMBODIMENTS

While the following is described in conjunction with specific embodiments, it will be understood that this description is intended to illustrate and not limit the scope of the preceding description and the appended claims.

A first embodiment of the invention is a process for conducting a carboxylate transfer reaction, the process comprising mixing an aromatic ring with a dicarboxylate alkali salt to form a mixture; and, heating the mixture in the presence of carbon dioxide to form a carboxylated aromatic compound and a decarboxylated alkali salt. An embodiment of the invention is one, any or all of prior embodiments in this paragraph up through the first embodiment in this paragraph, wherein the aromatic ring further comprises a counter ion, and wherein the alkali base, the counter ion, or both are selected from a group consisting of lithium, sodium, potassium, rubidium, cesium, and mixtures thereof. An embodiment of the invention is one, any or all of prior embodiments in this paragraph up through the first embodiment in this paragraph, wherein the dicarboxylate alkali salt comprises a 1, 3-dicarboxylate alkali salt. An embodiment of the invention is one, any or all of prior embodiments in this paragraph up through the first embodiment in this paragraph, wherein the mixture is heated to a temperature between 120° C. to 400° C. at a pressure up to 6,895 kPa (1,000 psi). An embodiment of the invention is one, any or all of prior embodiments in this paragraph up through the first embodiment in this paragraph, wherein the mixture comprises a slurry. An embodiment of the invention is one, any or all of prior embodiments in this paragraph up through the first embodiment in this paragraph, wherein the slurry is formed in a hydrocarbon. An embodiment of the invention is one, any or all of prior embodiments in this paragraph up through the first embodiment in this paragraph, wherein the carbon dioxide is provided as bubbles which flow counter current. An embodiment of the invention is one, any or all of prior embodiments in this paragraph up through the first embodiment in this paragraph, wherein the aromatic ring comprises a furoate, and wherein the dicarboxylate alkali salt is malonate. An embodiment of the invention is one, any or all of prior embodiments in this paragraph up through the first embodiment in this paragraph, further comprising regenerating the dicarboxylate alkali salt from the decarboxylated alkali salt.

A second embodiment of the invention is a process for producing a carboxylated aromatic compound, the process comprising passing an aromatic hydrocarbon and a dicarboxylate alkali salt to a vessel in a reaction zone to form a mixture; and, passing carbon dioxide into the vessel to contact the mixture; and, heating the mixture to form a carboxylated aromatic compound and a decarboxylated alkali salt. An embodiment of the invention is one, any or all of prior embodiments in this paragraph up through the second embodiment in this paragraph, wherein the aromatic hydrocarbon further comprises a counter ion, and wherein the alkali base, the counter ion, or both are selected from a group consisting of lithium, sodium, potassium, rubidium, cesium, and mixtures thereof. An embodiment of the invention is one, any or all of prior embodiments in this paragraph up through the second embodiment in this paragraph, wherein the dicarboxylate alkali salt comprises a 1, 3-dicarboxylate alkali salt. An embodiment of the invention is one, any or all of prior embodiments in this paragraph up through the second embodiment in this paragraph, wherein the aromatic hydrocarbon comprises a furoate, and wherein the dicarboxylate alkali salt is malonate. An embodiment of the invention is one, any or all of prior embodiments in this paragraph up through the second embodiment in this paragraph, wherein the mixture is heated to a temperature between 120° C. to 400° C. at a pressure up to 6,895 kPa (1,000 psi). An embodiment of the invention is one, any or all of prior embodiments in this paragraph up through the second embodiment in this paragraph, wherein the mixture comprises a slurry. An embodiment of the invention is one, any or all of prior embodiments in this paragraph up through the second embodiment in this paragraph, wherein the slurry is formed in a hydrocarbon. An embodiment of the invention is one, any or all of prior embodiments in this paragraph up through the second embodiment in this paragraph, wherein the carbon dioxide is provided as bubbles which flow counter current to the slurry. An embodiment of the invention is one, any or all of prior embodiments in this paragraph up through the second embodiment in this paragraph, further comprising separating the carboxylated aromatic compound from the hydrocarbon forming the slurry. An embodiment of the invention is one, any or all of prior embodiments in this paragraph up through the second embodiment in this paragraph, further comprising recycling the hydrocarbon forming the slurry. An embodiment of the invention is one, any or all of prior embodiments in this paragraph up through the second embodiment in this paragraph, further comprising regenerating the dicarboxylate alkali salt from the decarboxylated alkali salt.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for conducting a carboxylate transfer reaction, the process comprising:
mixing an aromatic ring with a dicarboxylate alkali salt to form a mixture; and, heating the mixture in the presence of carbon dioxide to form a carboxylated aromatic compound and a decarboxylated alkali salt.

2. The process of claim 1, wherein the aromatic ring further comprises a counter ion, and wherein the dicarboxylate alkali base, the counter ion, or both are selected from a group consisting of: lithium, sodium, potassium, rubidium, cesium, and mixtures thereof.

3. The process of claim 1, wherein the dicarboxylate alkali salt comprises a 1, 3-dicarboxylate alkali salt.

4. The process of claim 1, wherein the mixture is heated to a temperature between 120° C. to 400° C. at a pressure up to 6,895 kPa (1,000 psi).

5. The process of claim 1, wherein the mixture comprises a slurry.

6. The process of claim 5, wherein the slurry is formed in a hydrocarbon.

7. The process of claim 1, wherein the carbon dioxide is provided as bubbles which flow counter current.

8. The process of claim 1, wherein the aromatic ring comprises a furoate, and wherein the dicarboxylate alkali salt is malonate.

9. The process of claim 1, further comprising:
regenerating the dicarboxylate alkali salt from the decarboxylated alkali salt.

10. A process for producing a carboxylated aromatic compound, the process comprising:
passing an aromatic hydrocarbon and a dicarboxylate alkali salt to a vessel in a reaction zone to form a mixture; and,
passing carbon dioxide into the vessel to contact the mixture; and,
heating the mixture to form a carboxylated aromatic compound and a decarboxylated alkali salt.

11. The process of claim 10, wherein the aromatic hydrocarbon further comprises a counter ion, and wherein the dicarboxylate alkali base, the counter ion, or both are selected from a group consisting of: lithium, sodium, potassium, rubidium, cesium, and mixtures thereof.

12. The process of claim 10, wherein the dicarboxylate alkali salt comprises a 1, 3-dicarboxylate alkali salt.

13. The process of claim 10, wherein the aromatic hydrocarbon comprises a furoate, and wherein the dicarboxylate alkali salt is malonate.

14. The process of claim 10, wherein the mixture is heated to a temperature between 120° C. to 400° C. at a pressure up to 6,895 kPa (1,000 psi).

15. The process of claim 10, wherein the mixture comprises a slurry.

16. The process of claim 15, wherein the slurry is formed in a hydrocarbon.

17. The process of claim 15, wherein the carbon dioxide is provided as bubbles which flow counter current to the slurry.

18. The process of claim 15, further comprising:
separating the carboxylated aromatic compound from the hydrocarbon forming the slurry.

19. The process of claim 18, further comprising:
recycling the hydrocarbon forming the slurry.

20. The process of claim 10, further comprising:
regenerating the dicarboxylate alkali salt from the decarboxylated alkali salt.
